(19) United States
(12) Patent Application Publication
(10) Pub. No.: US 2021/0123905 A1
(43) Pub. Date: Apr. 29, 2021

(54) EVALUATION OF HYDROLYZED ALLERGEN PREPARATIONS

(71) Applicant: ASIT BIOTECH SA, Angleur (BE)

(72) Inventors: Cecile Pâques, Lierneux (BE); Thierry Legon, Roosbeek (BE); Sabine Pirotton, Bruxelles (BE); Nicolas Bovy, Grâce-Hollogne (BE)

(73) Assignee: ASIT BIOTECH SA, Angleur (BE)

(21) Appl. No.: 17/052,099

(22) PCT Filed: Apr. 30, 2019
(86) PCT No.: PCT/EP2019/061126

§ 371 Date: Oct. 30, 2020

(30) Foreign Application Priority Data
Apr. 30, 2018 (EP) 18170129.3

(51) Int. Cl. G01N 33/50

(52) U.S. Cl. CPC G01N 33/5052

(57) ABSTRACT

A method for the evaluation of a hydrolyzed allergen preparation comprising the steps of: bringing the preparation into contact with a human blood sample—measuring proliferation of IL10 producing regulatory B-cells, wherein proliferation indicates suitability of the preparation.

The present invention is related to the evaluation of hydrolyzed allergen preparations.

The only available solution of disease modifying effect in allergy (type I hypersensitivity) is immunotherapy. This is achieved by repeated administration of the culprit allergen which involves risk of allergic reaction. One solution to reduce side effects and treatment duration is the use of hydrolyzed allergens which have become a more and more promising active principle for the induction of tolerance in allergic patients; see WO 2008/000783 and WO 2012/172037.

The major advantage of allergen hydrolysis into peptides (allergen fragments) is the reduction of allergenicity and consequently the risk of systemic reaction. However, the conservation of all information necessary for immune reprogramming in the active principle should be conserved. Therefore, a preparation that is not sufficiently hydrolyzed might have a higher allergenicity and immunogenicity but might also be more risky during administration while a preparation that is hydrolyzed too much would no longer be effective. The balance between safety and efficacy is then crucial for the selection of the best product candidate for allergy immunotherapy.

In the process of developing tolerance following immunotherapy, IL10 cytokine is a key player in the reprogramming of the immune system. Several studies showed that IL10 producing T cells are essential for efficient immunotherapy in IgE-mediated allergy. Peptide immunotherapy in cat-allergic and rheumatoid arthritis (RA) patients has been shown to induce IL10 from T cell subsets and higher levels of IL10 in culture supernatants (Campbell J D, Buckland K F, McMilIan S J, Kearley J, Oldfield W L G, Stern L J, et al. in J Exp Med. 2009; 206(7):1535-47; Prakken B J, Samodal R, Le T D, Giannoni F, Yung G P, Scavulli J, et al. Proc Natl Acad Sci USA 2004; 101(12):4228-33; Verhoef A, Alexander C, Kay A B, Larché M. PLoS Med. 2005; 2(3):0253-61).

Murine studies of HDM peptide immunotherapy have also shown an upregulation of CD4⁺IL10⁺ but not CD19⁺ B-cells (Moldaver D M, Bharhani M S, Wattie J N, Ellis R, Neighbour H, Lloyd C M, et al. Br Dent J. 2014; 217(2):379-90). These studies have clearly demonstrated that the IL10 production following peptide immunotherapy are mainly IL10⁺ T but not B cell-driven. These IL10⁺ T-cells have the ability to downregulate the antigen-specific Th2 responses with concomitant increase in regulatory cytokines such as IL10.

On the other hand, regulatory B-cells play a role in immunological tolerance; see for example E. C. Rosser and C. Mauri, Immunity 42 (2015) 607-612 or C. Mauri and M. R. Ehrenstein, TRENDS in Immunology 29 (2007) 34-40. Activated B-cells can also secrete IL10 when activated through TLR4 and TLR9 agonists in the presence of CD40L. Moreover, IL10 producing B cells are mainly known to be involved in the induction of tolerance of another type of hypersensitivity (type IV) namely non IgE-mediated allergy. The latter, compared to IgE-mediated allergy, is typically delayed with symptoms appearing hours to weeks after exposure. It also involves different immunological mechanisms with activation of Th1 response instead of Th2 response leading to over-activation of macrophages and inflammation.

S. J. Lee et al. in Allergy Asthma Immunol Res. 5 (2013) 48-54 discloses in-vitro induction of allergen-specific IL10 producing regulatory B cell responses by interferon-gamma in non-IgE-mediated milk allergy.

J. Noh at al. in Cellular Immunology 273(2012) 140-149 discloses tolerogenic effects of interferon-gamma with induction of allergen-specific interleukin-10-producing regulatory B cell changes in non-IgE-mediated food allergy.

Unexpectedly, it was found that peptides were able to generate de novo IL10+ B-cells (CD19⁺CD27⁺IL10⁺, CD19⁺CD5⁺CD24ⁱⁿtCD38^{int}, CD19⁺CD5⁺IL10⁺ B-cells) whilst also activating constitutively expressing IL10⁺ Bregs subset (CD19⁺CD5⁺CD24^{hi}CD38^{hi}) as mechanism of tolerance in the context of IgE-mediated allergy. These are novel findings for type I hypersensitivity, which have not been previously described in literature and challenges the current dogma how to understand the mechanisms of peptide immunotherapy.

The object of the present invention is to provide a method for the evaluation of hydrolyzed allergen preparations. This object is solved by a method for the evaluation of a hydrolyzed allergen preparation comprising the steps of:
bringing the preparation into contact with a human blood sample
measuring proliferation of IL10 producing regulatory B-cells,
   wherein proliferation indicates suitability of the preparation.

According to the method of the invention, the hydrolyzed allergen preparation is brought into contact with a blood sample. This is performed in vitro.

It is then measured if IL10 producing regulatory B-cells proliferate upon contact, wherein proliferation indicates the suitability of the preparation. Suitability in this context means that it is suitable for the future use. It indicates in some way a quality of the preparation. Therefore, in one embodiment the method is used as a quality control within a production process.

In an alternative embodiment, it may be used for screening and drug development. In this embodiment a batch of hydrolyzed allergen preparation is prepared. Depending on the results of evaluation, the process may be slightly modified to improve the product. For example, the amount of enzyme used for the hydrolysis, the time of hydrolysis, the temperature or the concentration of the allergen may be modified.

In one embodiment the method further comprises
bringing an unhydrolyzed allergen preparation into contact with a second blood sample
measuring proliferation of IL10 producing regulatory B-cells,
comparing proliferation in the two samples,
   wherein a higher proliferation in the sample being in contact with the hydrolyzed allergen preparation than in the sample in contact with the unhydrolyzed allergen indicates suitability.

In this embodiment, two blood samples of the same subject are brought into contact with two forms of the allergen, one with in the allergen in hydrolyzed form and one in the unhydrolyzed form. The amounts of allergens are the same in both preparations.

By comparing the proliferation in the two samples, the suitability of the preparation can be further tested. A suitable preparation shows a higher proliferation of the IL10 producing regulatory B-cells in contact with hydrolyzed preparations than with the unhydrolyzed allergen

While in some embodiments, the blood samples may be from a subject that is allergic to the specific allergen, in a preferred embodiment the blood sample is from a subject that is not allergic to the allergen or is non-allergic to typical allergens at all.

Suitable allergens used according to the invention are selected among plant allergens, pollen allergens, milk allergens, venom allergens, egg allergens, weed allergens, grass allergens, tree allergens, shrub allergens, flower allergens, vegetable allergens, grain allergens, fungi allergens, fruit allergens, berry allergens, nut allergens, seed allergens, bean allergens, fish allergens, shellfish allergens, seafood allergens, meat allergens, spices allergens, insect allergens, mite allergens, mould allergens, animal allergens, pigeon tick allergens, worm allergens, soft coral allergens, animal dander allergens, nematode allergens, allergens of Hevea brasiliensis.

In some embodiments the method comprises further steps of preparing hydrolyzed allergens.

In one embodiment, the method comprises the steps:
a) extracting a natural source of allergens comprising allergenic proteins to form an extract,
b) purifying of said extract to remove non-protein components to form a purified extract,
c) denaturing said purified extract to form a purified denatured extract,
d) hydrolyzing the purified denatured extract to form hydrolyzed allergen peptides.

In other embodiments, the method may comprise:
a) extracting a source of allergens comprising allergenic proteins to form an extract,
b) purifying the extract to remove non-protein components to form a purified extract,
c) denaturing the purified extract with a first denaturing agent to form a purified denatured extract,
d) refining the purified denatured extract to remove impurities to form a refined denatured extract,
e) denaturing the refined denatured extract with a second denaturing agent to form denatured allergen mixture, and
f) hydrolyzing the denatured allergen mixture to form the hydrolyzed allergen peptides.

Preferred embodiments include methods wherein the IL10 producing regulatory B-cells are
CD19⁺ IL10⁺ B-cells.
CD19⁺ CD27⁺ IL10⁺ B-cells.
CD19⁺ CD5⁺ CD38^{hi}CD24^{hi}IL10⁺ B-cells.
CD19⁺ CD5⁺ CD38^{int}CD24^{int}IL10⁺ B-cells.

### DESCRIPTION OF FIGURES

FIGS. 1A, 1B and 1C show the SEC data of threes grass pollen hydrolyzate batches.

FIGS. 2A, 2B and 2C show the kinetic of production of grass pollen sIgG (A), peanut sIgG (B) and house dust mite sIgG (C).

FIGS. 3A, 3B and 3C show Antibody reactivity against Lolium perenne (A), Arachis hypogaea (B), and Dermatophagoides pteronyssinus (C) allergens by Western Blot.

FIG. 4 shows Allergenicity - Facilitated Antigen Binding (FAB) for grass pollen allergics compared to non-atopic controls.

FIGS. 5A, 5B and 5C show Allergenicity—Basophil Activation Test (BAT) by grass pollen (A), peanut (B) and house dust mite (C) preparations for allergics compared to non-atopic controls.

FIGS. 6A, 6B and 6C show Immunogenicity—Induction of CD19⁺ IL10⁺ B-cells by grass pollen (A), peanut (B) and house dust mite (C) preparations for allergics compared to non-atopic controls.

FIGS. 7A, 7B and 7C show Immunogenicity—Induction of CD19⁺ CD27⁺ IL10⁺ B-cells by grass pollen (A), peanut (B) and house dust mite (C) preparations for allergics compared to non-atopic controls.

FIGS. 8A, 8B and 8C show Immunogenicity—Induction of CD19⁺ CD5⁺ CD38^{hi} CD24^{hi}IL10⁺ B-cells by grass pollen (A), peanut (B) and house dust mite (C) preparations for allergics compared to non-atopic controls.

FIGS. 9A, 9B and 9C show Immunogenicity—Induction of CD19⁺ CD5⁺ CD38^{int} CD24^{int} IL10⁺ B-cells by grass pollen (A), peanut (B) and house dust mite (C) preparations for allergics compared to non-atopic controls.

All references cited herein are incorporated by reference to the full extent to which the incorporation is not inconsistent with the express teachings herein.

The invention is further explained by the following, non-limiting examples.

### EXAMPLES

### Example 1

### Preparation of Grass Pollen (Lolium perenne) Peptides

### Example 1.1

### Extraction

1% (w/v) pollen (Lolium perenne from ALLERGON) was added to sodium bicarbonate (12.5 mM) and incubated 2 h under stirring. The solution was then clarified and filtrated by adding celite (ACROS) at 2% (w/v) and passing through a 0.2 μm filter. This sample constitutes the crude extract.

The presence of allergens in the extract was analyzed by western blotting using pollen allergic patient sera. IgG and IgE epitopes are visualized with anti-human IgG or IgE antibodies.

The said crude extract was acidified to pH 3.0 and Tween 20 (0.1%, v/v) was added. This sample constitutes the acidified extract.

### Example 1.2

### Purification of Allergen Proteins

The allergen extract was purified by:

Cation exchange chromatography
A sartobind S⁻ membrane (SARTORIUS) was equilibrated with 28× Bed volume (Bv) of sodium bicarbonate 12.5 mM, citrate 30 mM, pH 3.0, Tween 20 0.1% (v/v). The said acidified extract was loaded on the equilibrated membrane. The column was washed first with 35× By of sodium bicarbonate 12.5 mM, citrate 30 mM, pH 3.0, Tween 20 0.1% (v/v) and then washed with 42× Bv of sodium bicarbonate 12.5 mM, citrate 30 mM, pH 3.0. The proteins were eluted with carbonate 0.1 M, sodium chloride 0.5 M, pH 9.15. The presence of proteins was followed the OD at 280 nm. The fractions of interest were pooled.

Ammonium sulfate precipitation
This step was performed at 0-4° C.
A quantity of ammonium sulfate to reach 90% of saturation was added to the product under stirring. The stirring was stopped after the complete dissolution of the salt. The suspension was incubated overnight and centrifuged 2 times during 15 min at 10,000 g. The supernatant was each time carefully discarded.

Denaturation
The pellets were resuspended at 9 mg/ml in urea 6 M, DTT 10 mM, Tris.HCl 0.1 M, pH 8.0 and incubated at 37° C. for 1 h.

Size exclusion chromatography on G25 resin (fine Sephadex from AMERSHAM)
The denatured sample was loaded on the column and the proteins were eluted with Tris.HCl 25 mM, urea 1.5 M, pH 8.0.

The presence of proteins was followed by the OD measurement at 280 nm The fractions of interest were pooled to constitute the purified denaturated allergen extract.

The purified allergen extract was further analyzed. The protein content (BCA Assay) and the dry weight were determined in order to evaluate the protein purity. The purification efficiency was also followed by the removal of carbohydrates (Orcinol test) and by the decrease of the ratio OD₂₆₀/OD₂₈₀.

TABLE 1
Removal of non-protein components to form a purified extract

| | Ratio protein/ | Ratio OD₂₆₀/ | Ratio carbohydrates/ |
|---|---|---|---|
| | dry weight | OD₂₈₀ | proteins |
| Crude extract | 16% | 1.3 | 400% |
| Purified extract | 85% | 0.75 | 17% |

As shown in table 1, the purification process allows

The increase of the percentage of proteins in the extract from ˜15% to 80%

The OD₂₆₀/OD₂₈₀ ratio to tends towards 0.5 characterizing a pure protein

A significant removal of carbohydrates (the residual content could represent the carbohydrate moiety of the proteins).

### Example 1.3

### Hydrolysis of Denatured Allergen Extract

The extract was hydrolyzed using the following protocol:

The said purified allergen extract was acidified to pH 2.0. The digestion was performed at 2.5 mg/ml of pollen proteins and 1 Eu. Ph. U of pepsin (MERCK) for 337 mg of proteins, at 37° C., during 2 h.

### Example 1.4

### Purification

In order to eliminate the peptides with a MW≥10,000 Da and MW≤1,000 Da, the hydrolyzate was purified by

Size exclusion chromatography on G50 resin (fine Sephadex from AMERSHAM)
16.5% (v/v) of isopropanol and 0.1 M of NaCl were added to the hydrolyzate. This sample was immediately loaded on a G50 column. The peptides were eluted and the fractions containing the peptides (MW≤10 kDa) were pooled 6.

Diafiltration on 1kDa membrane (ultrafiltration cassette Omega PES from PALL)
The peptides were concentrated 10×, diafiltrated against 10 volumes of Tris.HCl 50 mM pH 7.4 and finally concentrated 2.5×. This sample constitutes the purified allergen hydrolyzate.

The efficiency of the purification was controlled by size exclusion HPLC. A BioSep-SEC S2000 column (PHENOMENEX) was equilibrated with Na2HPO₄ 50 mM—SDS 0.5% (w/v) pH 6.8 at a flow rate of 1 ml/min. The peptides were detected at 214 nm.

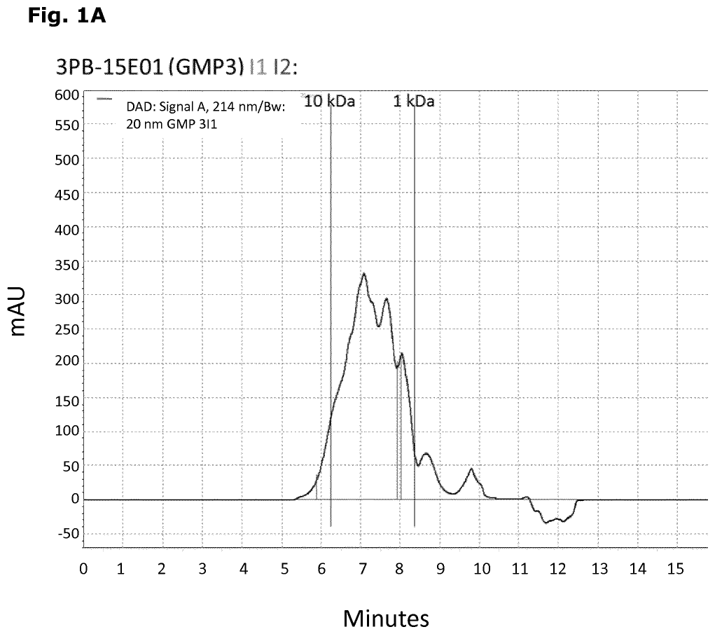

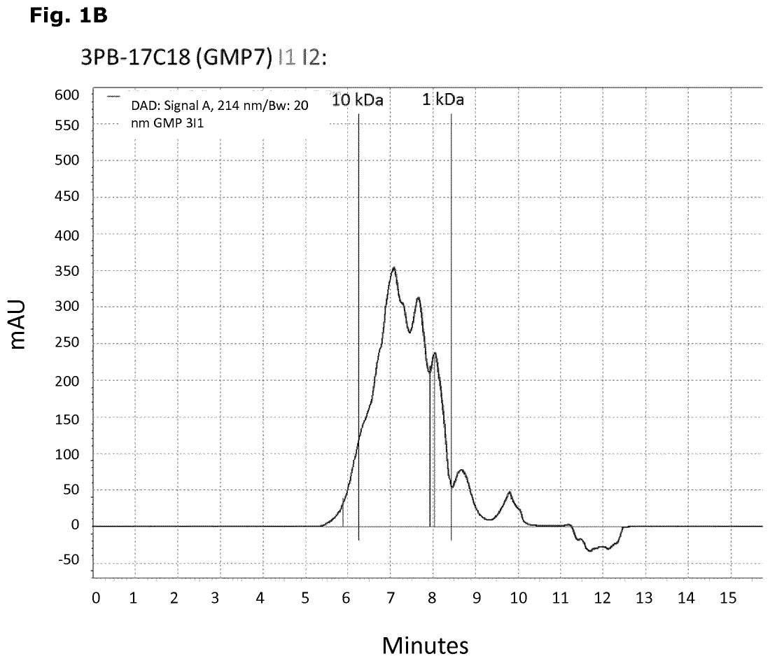

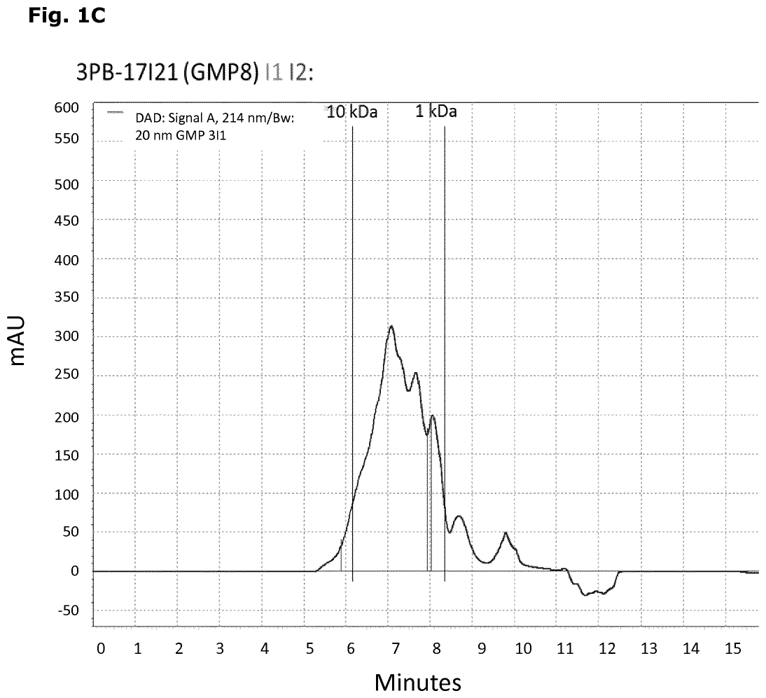

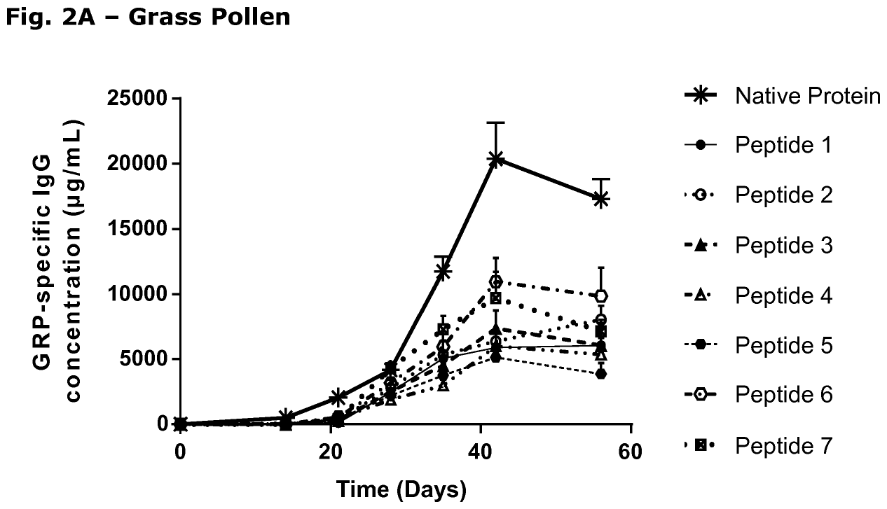

Three examples of size exclusion chromatography are shown in FIG. 1.

### Example 2

### Preparation of Peanut (Arachis hypogaea) Peptides

### Example 2.1

### Extraction of Peanut Allergens

A mix of three peanut types (Arachis hypogaea species Runner, Virginia and Spanish) were peeled, grinded and mixed. A 2% (w/v) of the mix of peanuts was added to sodium phosphate (12.5 mM) and incubated 1 h under stirring at room temperature. The solution was then clarified and filtrated by adding Celite at 2% (w/v) and passing through a 0.45 μm filter. This sample constitutes the crude protein extract.

The presence of allergens in the crude protein extract was confirmed by Western-Blot using peanut allergic patient sera.

### Example 2.2

### Purification of Peanut Allergen Proteins

The allergen extract was purified by:
Trichloroacetic acid precipitation

This step was performed at room temperature (20 to 25° C.).

10% (w/v) trichloroacetic acid was added to the product under stirring. Then, the precipitated extract was centrifuged during 15 minutes at 10.000 g. The supernatant was carefully discarded.
First Denaturation

The pellets were resuspended at 25 mg/ml in 8 M Urea, 0.1 M Tris-HCl, pH 8.0 and 80 mM DTT were added. The solution was incubated at 37° C. for 1 h.
Size exclusion chromatography on a G25 resin column (fine Sephadex from GE Healthcare)

The purified denatured extract was immediately loaded on the column and the proteins were eluted with 2 M Urea, 0.1 M Tris-HCl, pH 8.0.

The presence of proteins was followed by the absorbance at 280 nm. The fractions of interest were pooled to constitute the refined denatured extract.

The refined denatured extract was further analyzed by SDS-PAGE and by Western Blotting using peanut allergic patient sera.
Second Denaturation:

8 M urea and 40 mM TCEP were added to the refined denatured extract. Then, the pH was adjusted to 2.5. The solution was incubated at 37° C. for 1 h.

### Example 2.3

### Hydrolysis of the Denatured Peanut Allergens

The denatured allergens were hydrolyzed using the following protocol:

The denatured allergen mixture was diluted 4-fold with 10 mM HCl and acidified with HCl 6 N to pH 2.0. The protein hydrolysis was performed with 16 Eu.Ph.0 of pepsin for 100 mg of proteins at 37° C., during 2 h. The hydrolysis was then stopped by raising the pH to 10.0 with NaOH solution.

### Example 2.4

### Purification of Hydrolyzed Peanut Allergens

In order to eliminate the peptides with a MW 10.000 Da and MW 1.000 Da, the hydrolyzed allergens were purified by:
Size exclusion chromatography on G50 resin (fine Sephadex from GE Healthcare). After increasing pH, the hydrolyzed allergens were rapidly loaded on the G50 column. The peptides were eluted with 2 M Urea, 0.1 M Tris-HCl, pH 9.5. The elution was followed by the absorbance at 280 nm. The fractions containing the peptides (MW 10 kDa) were.
Diafiltration on 1 kDa membrane (ultrafiltration cassette Omega PES from PALL). The peptides were concentrated 25-fold, diafiltrated against 10 volumes of 50 mM sodium phosphate at pH 7.6 and finally concentrated 2-fold. This sample constitutes the purified hydrolyzate.

The purified hydrolyzate was analyzed by SDS-PAGE. The profile shows that there are no residual proteins with molecular weights above 10 kDa.

The efficiency of the purification was controlled by size exclusion HPLC. A BioSep-SEC S2000 column was equilibrated with 50 mM Na₂HPO₄, 0.5% (w/v) SDS, pH 6.8 at a flow rate of 1 ml/min. The peptides were detected at 215 nm.

### Example 3

### Preparation of House Dust Mite (Dermatophagoides pteronyssinus) Peptides

### Example 3.1

### Protein extraction of House Dust Mite

Proteins from House Dust Mite were extracted by incubation in Phosphate Buffer Saline pH 7.4 during 1 h at room temperature under stirring. The solution was clarified and filtrated by adding Celite at 2% (w/v) and passing through a 0.45 μm PVDF filter. This sample constitutes the crude protein extract.

The crude protein extract seems to show the major allergens (Derp1, Derp2) which can be localized according to their molecular weight (25 kDa and 14 kDa respectively).

### Example 3.2

### Purification of Allergen Proteins from House Dust Mite

The purification was performed by:
Trichloracetic acid precipitation

10% (w/v) trichloracetic acid was added to the crude protein extract under stirring for 5 min at room temperature. The proteins were collected by centrifugation during 20 min at 10.000 g.
First denaturation

After elimination of the supernatant, the pellet was resuspended in 8 M urea, 0.1 M Tris pH 7-8. The solution was incubated for 1 h at 37° C. after pH adjustment to 7.5 and addition of 80 mM DTT.
Size exclusion chromatography on G25 resin column

The proteins from the denaturated extract were loaded on the column, and eluted with 2 M Urea, 0.1 M NaCl pH 9.0.

The presence of proteins was monitored by the measurement of the absorbance at 280 nm.
Second denaturation

The denaturation occurred by incubation at 37° C. for 1 h in 4 M urea, 0.1 M NaCl and 40 mM TCEP with the pH adjusted to 2.5.

### Example 3.3

### Hydrolysis of the Denaturated Allergens for House Dust Mite

The denaturated protein mixture was previously diluted 2-fold with 10 mM HCl and acidified with HCl 6N to pH 2.0. The hydrolysis of proteins was conducted with 16 Eu.Ph.0 of pepsin per 100 mg for 1 h at 37° C.

### Example 4

### Evaluation of Peptide Safety and Efficacy

### Example 4.1

### Production of sIgG Following Mice Immunization

Several batches of hydrolyzed allergens were prepared according to examples 1 to 3.

Groups of 8-10 mice were immunized with 6 intraperitoneal injections of 100 μg of different batches of allergen fragments combined with alum at a weekly interval. As positive control, one group of mice was immunized with unhydrolyzed full length allergens (proteins). Kinetic of specific IgG antibody production was measured by ELISA up to Day 56.

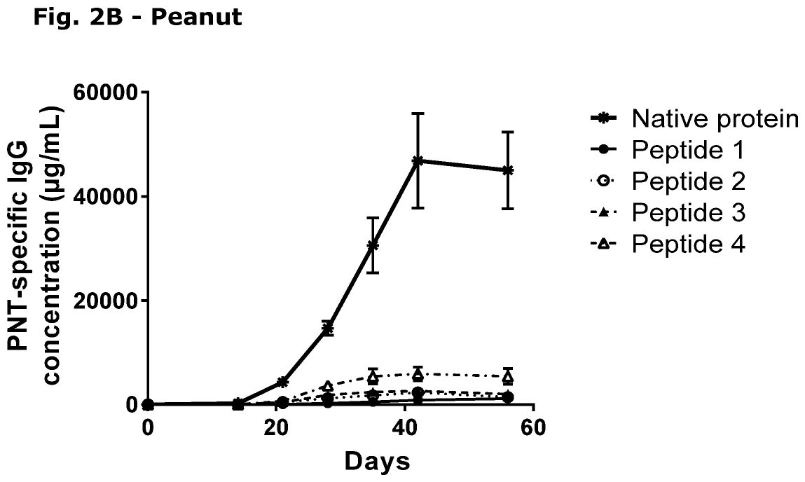

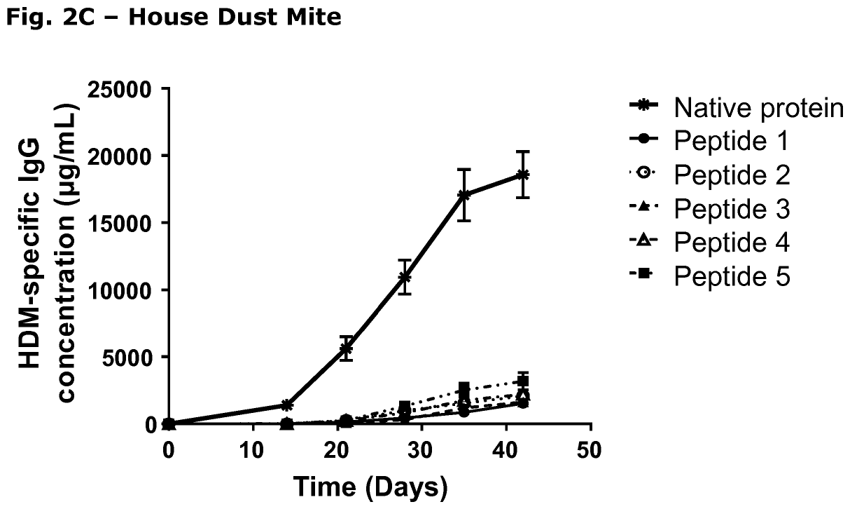

Results for grass pollen allergen (Lolium perenne) fragments are shown in FIG. 2A; peanut allergen (Arachis hypogaea) fragments in FIG. 2B; and house dust mite allergen (Dermatophagoides pteronyssinus) fragments in FIG. 2C. Data are presented as mean±SEM, n=10 per group. For the 3 types of allergens, all peptide batches were statistically different in terms of immunogenicity from the native proteins. In addition, there was no statistical differences between peptide batches.

### Example 4.2

### Antibody Reactivity Against Allergen Fragments

Serum from different groups of mice (as explained in example 4.1) was collected at Day 42 and evaluated for their reactivity against full length-allergens by Western Blotting analysis. Proteins were loaded on a SDS-polyacrylamide gel, submitted to electrophoresis and transferred on a PVDF membrane under electric field. The PVDF membrane was cut into pieces, one for each sample tested, and were incubated with the serum of one group of mice. Binding was detected by anti-mouse IgG coupled to biotin and revealed by streptavidin coupled to a fluorescent label (europium).

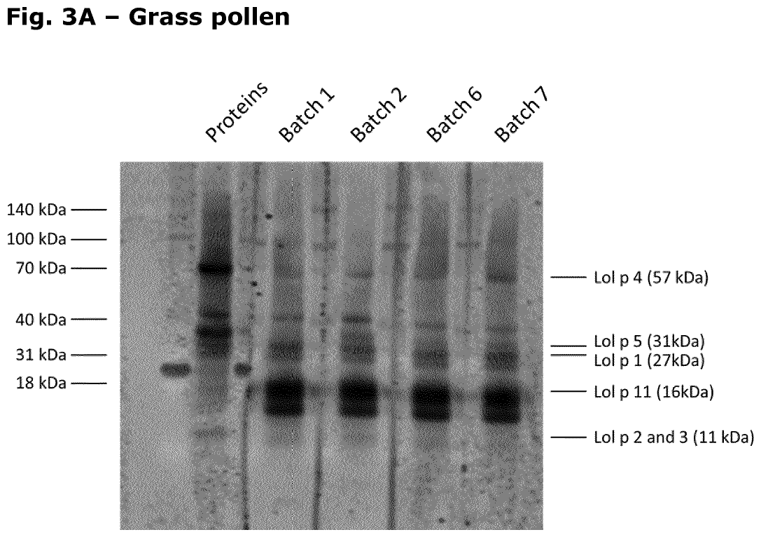

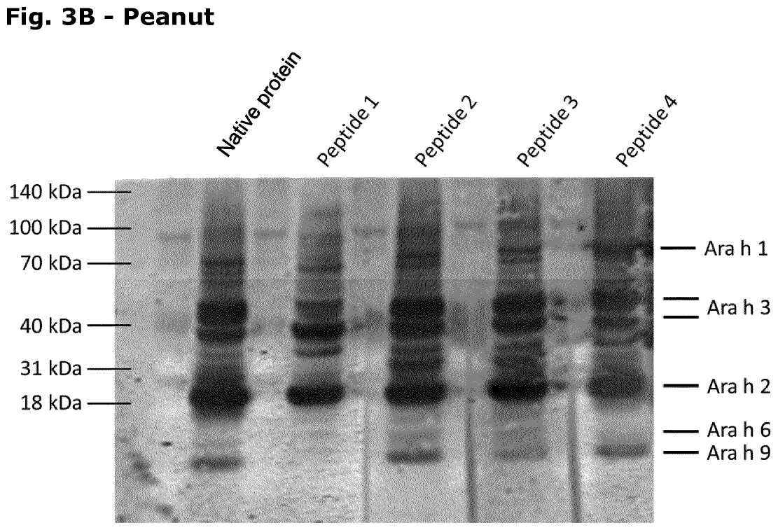

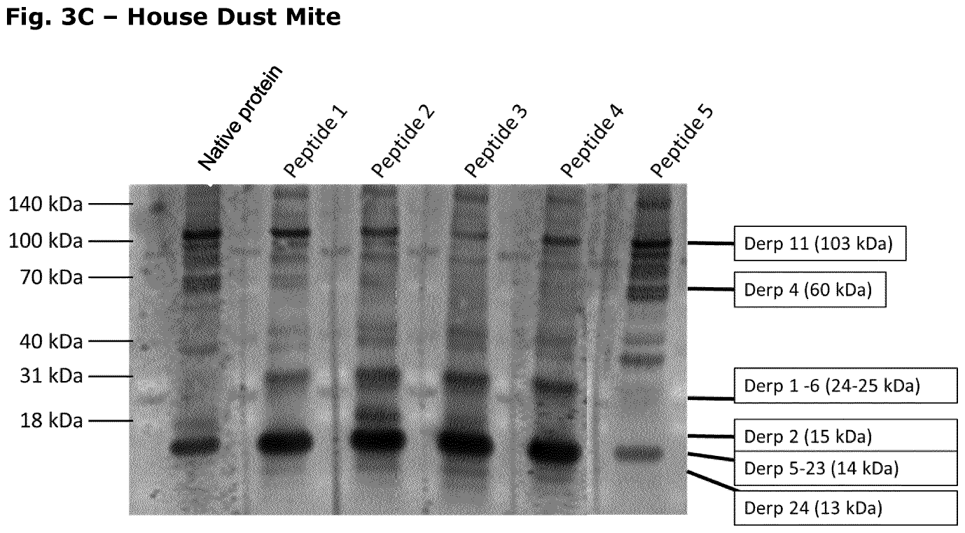

FIG. 3 displays results from mice immunized with grass pollen (Lolium perenne) (A), peanut (Arachis hypogaea) (B), and house dust mite (Dermatophagoides pteronyssinus) (C) proteins or allergen fragments. For all allergens, serum from peptide-immunized mice recognized full length-allergens. However, some variability can be observed between peptides as well as with the serum from the group immunized with undigested allergens.

### Example 4.3

### Allergenicity—Facilitated Antigen Binding (FAB)

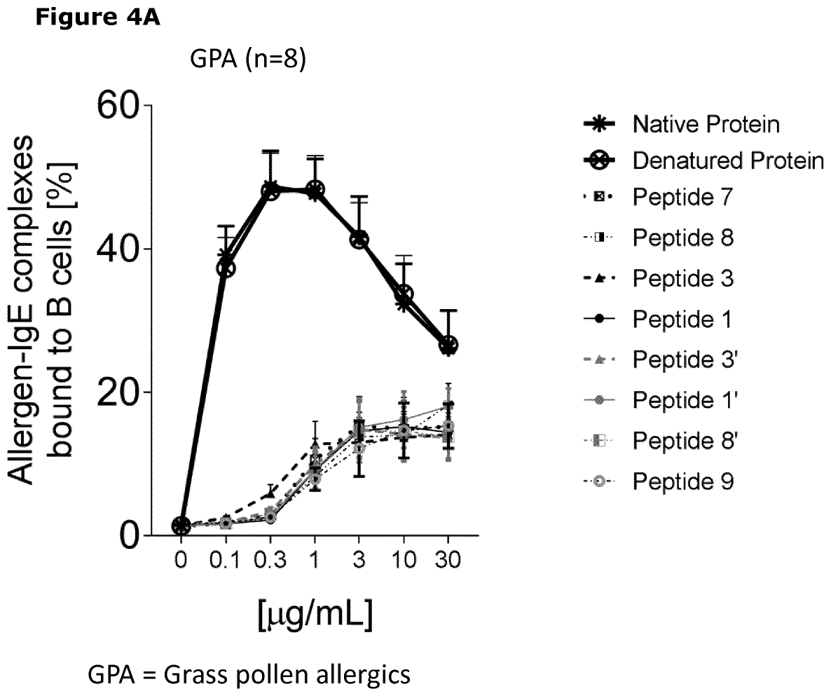

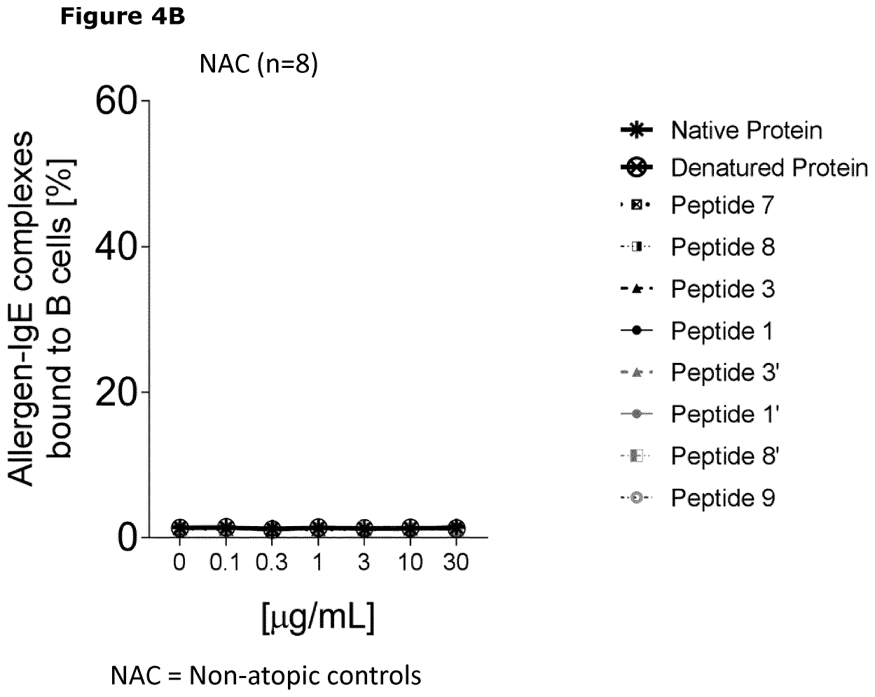

The allergenicity of various batches of allergen product was evaluated by IgE-facilitated allergen binding to B-cells as described in Shamji, M. H. et al. The IgE-facilitated allergen binding (FAB) assay: Validation of a novel flow-cytometric based method for the detection of inhibitory antibody responses. J. Immunol. Methods 317, 71-9 (2006). Serum from allergic (GPA, n=8) and non-atopic (NAC, n=8) subjects were pre-incubated with increasing concentrations of various product for 1 h at 37° C., followed by addition of 1×10⁵ EBV-transformed B-cells to allergen-IgE mixture and were further incubated for 1 h at 4° C. The allergen IgE complexes was determined by polyclonal human anti-IgE PE-labelled antibody and acquired by FACS. Results are shown in FIG. 4. A dose dependent binding of allergen-IgE complexes was observed in case of allergic subjects specifically, no binding was observed when blood sample from non-atopic subjects were used. In addition, unhydrolyzed allergens were more potent and more efficacious than peptides to induce the binding of complexes to B-cells.

### Example 4.4

### Allergenicity—Basophil Activation Test (BAT)

Allergenicity of various allergen batches from grass pollen, peanut and house dust mite fragments was determined by basophil activation test and diamine oxidase by flow cytometry.

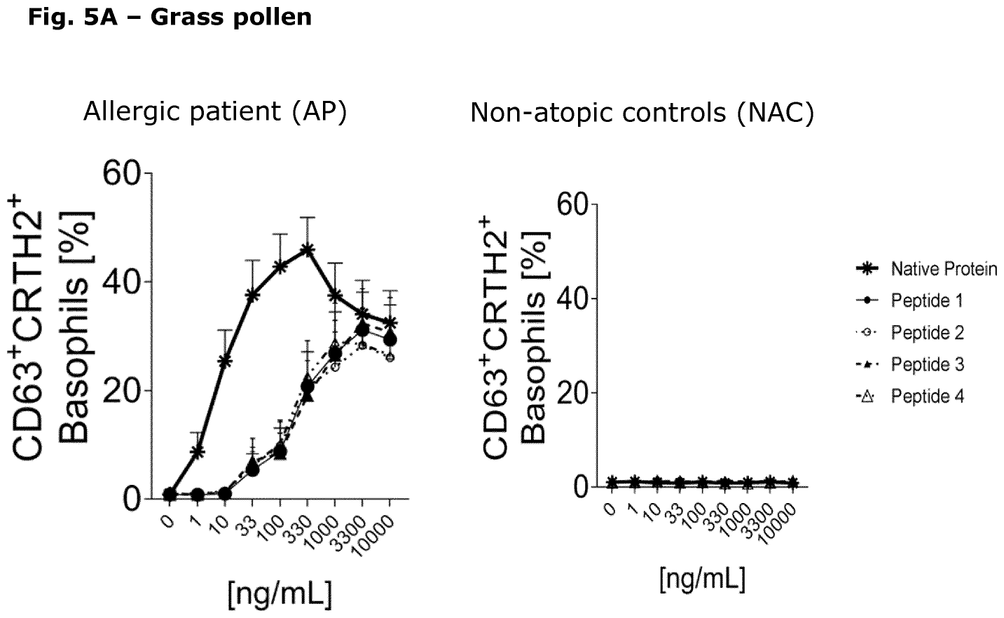

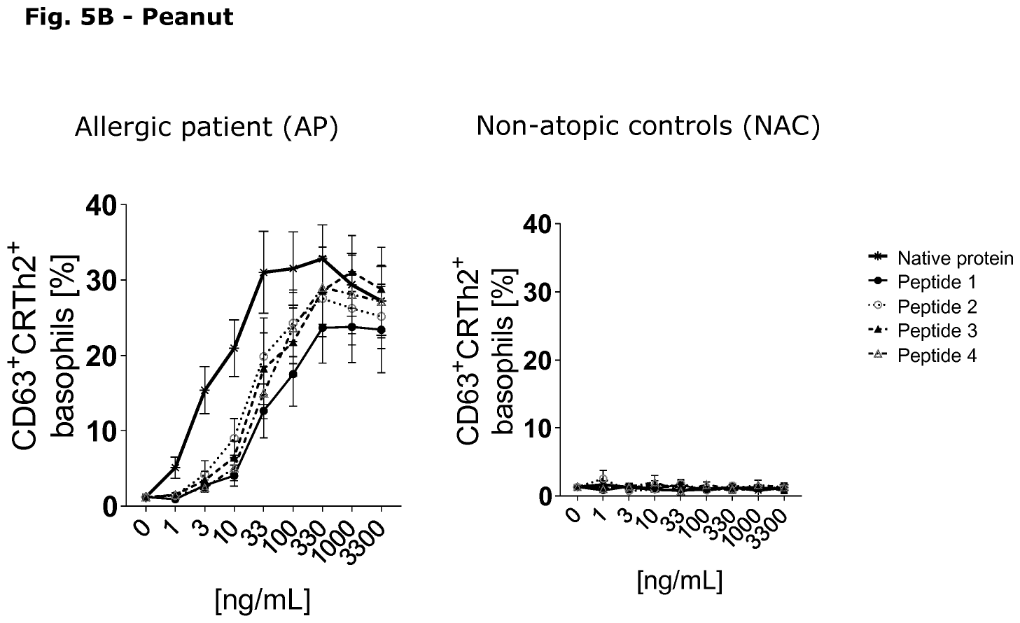

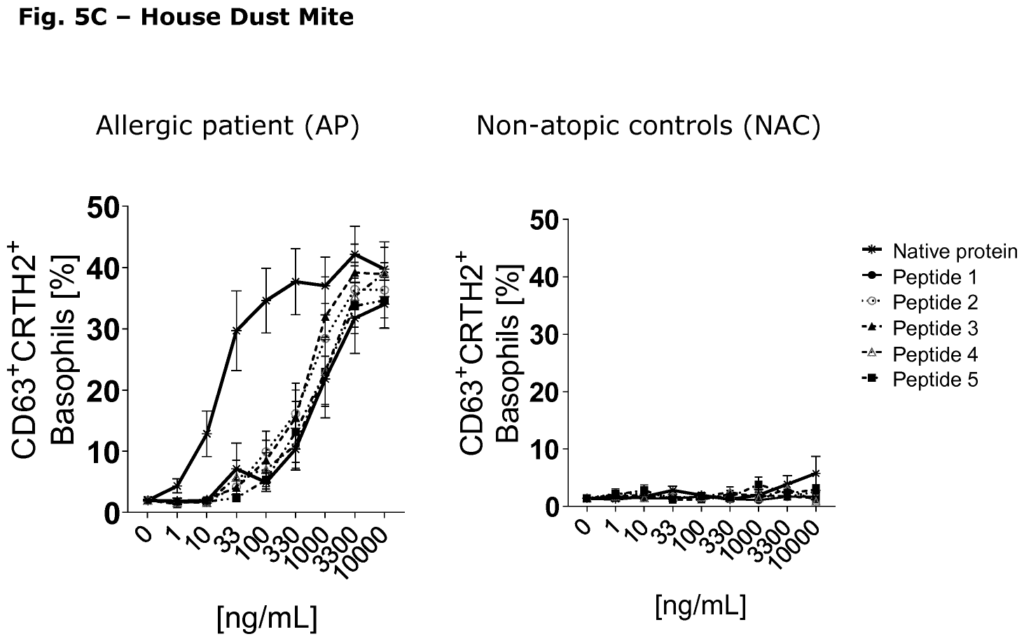

Whole blood from allergic (AP, n=16) and non-atopic (NAC, n=6) individuals was incubated with increasing concentrations of one batch of proteins (unhydrolyzed allergens) and different batches of allergen fragments. Basophil activation was measured using flow cytometric method of the expression of the CD63 marker on the cell membrane of activated cells. Results for grass pollen (Lolium perenne) allergen fragments are shown in FIG. 5A. Allergens induced basophil activation of allergic subjects specifically, no activation was observed in blood sample from non-atopic subjects. As shown in FIG. 5A, unhydrolyzed allergens were 20-40 times more potent to induce basophil degranulation than peptides.

Results for peanut (Arachis hypogaea) allergen fragments are shown in FIG. 5B. Allergens induced basophil activation of allergic subjects specifically, no activation was observed in blood sample from non-atopic subjects. As shown in

FIG. 5B, unhydrolyzed allergens were 5 times more potent to induce basophil degranulation than peptides

Results for house dust mite (Dermatophagoides pteronyssinus) allergen fragments are shown in FIG. 5C. Allergens induced basophil activation of allergic subjects specifically, no activation was observed in blood sample from non-atopic subjects. As shown in FIG. 5C, unhydrolyzed allergens were 10 times more potent to induce basophil degranulation than peptides

### Example 4.5

### Immunogenicity—Induction of CD19⁺IL10⁺ B-cells

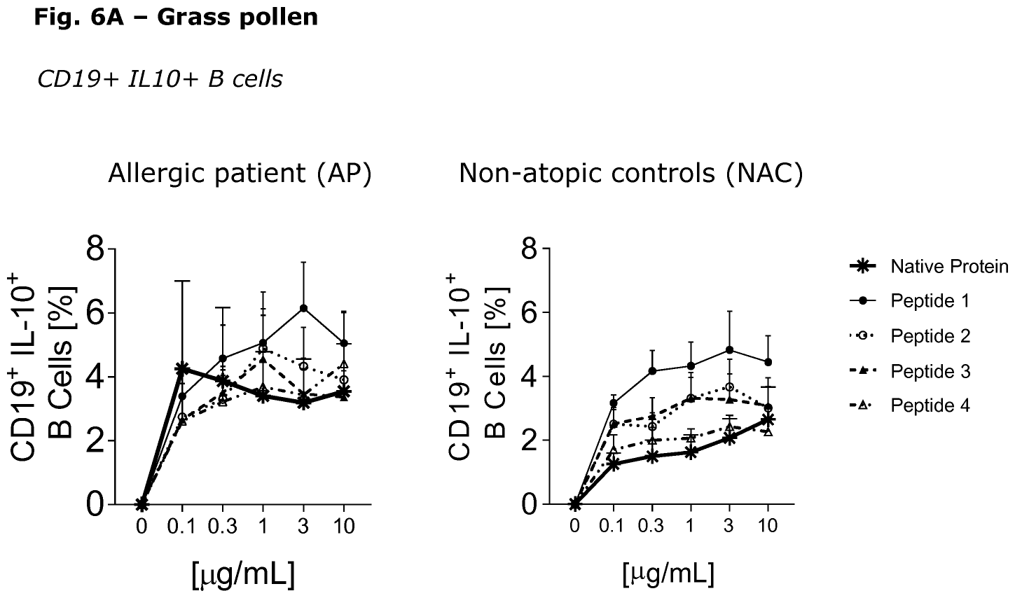

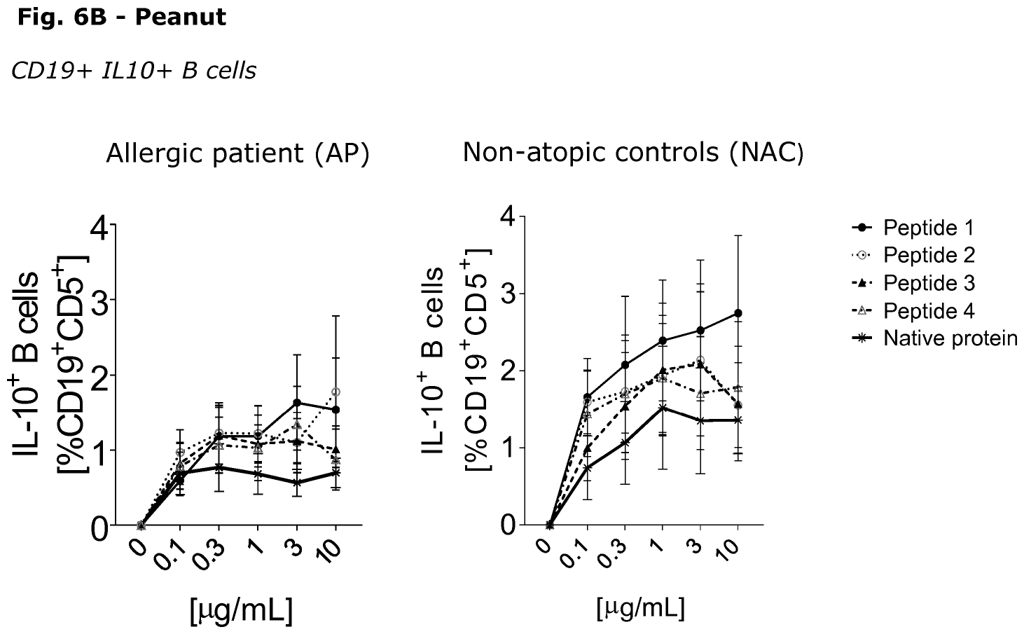

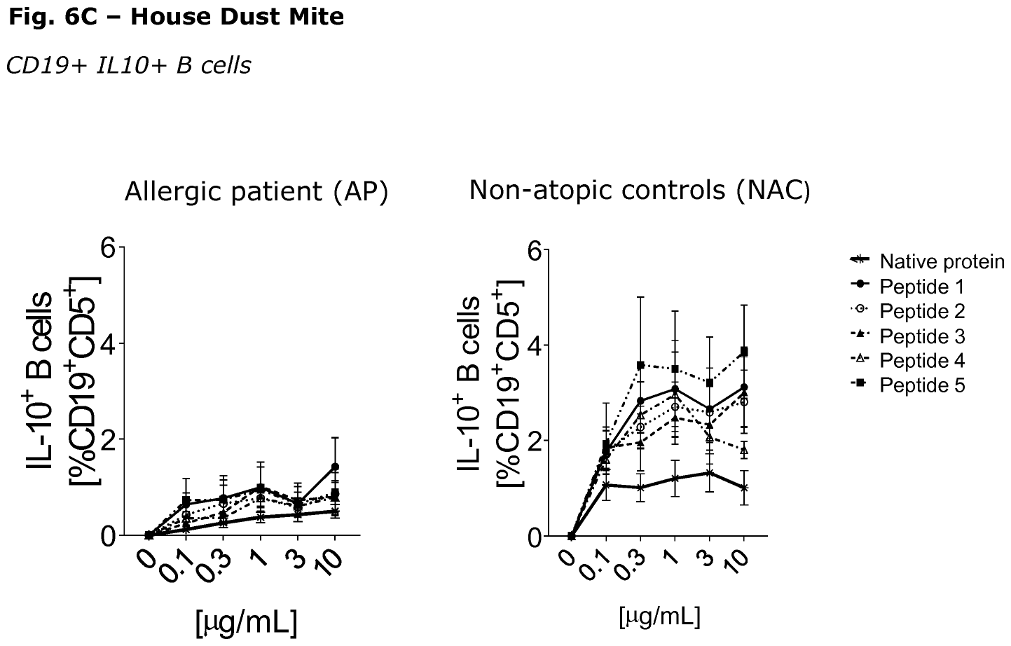

Effect of allergen fragments and unhydrolyzed allergens (proteins) was assessed on PBMCs isolated from allergic (AP, n=16) and non-atopic (NAC, n=6) individuals using flow cytometry. PBMCs were stimulated with 0, 0.1, 0.3, 1, 3 & 10 pg/mL concentrations of allergen fragments or full length allergens for 72 hrs at 37° C. Cells were stimulated with PMA, Ionomycin and BFA (Brefeldin A) and incubated for a total of 5 hrs at 37° C. Following incubation, cells were immunostained with CD19 for 30 min at room temperature. Cells were fixed and permeabilized using Cytofix/cytoperm reagent for 20 min at 4° C. and immunostained with IL10 for 30 min. Cells were washed and resuspended in cell staining buffer before acquisition on the BD FACS Canto II instrument. Results are shown in FIG. 6A for grass pollen (Lolium perenne) allergen fragments; FIG. 6B for peanut (Arachis hypogaea) allergen fragments and FIG. 6C for house dust mite (Dermatophagoides pteronyssinus) allergen fragments. For all 3 allergens, the proportion of CD19⁺IL10⁺ B-cells were significantly increased after stimulation with peptide batches compared to native proteins in non-atopic subjects. This increase was observed in CD19⁺IL10⁺ B-cells in a dose-dependent manner. A similar trend was observed in allergic subjects.

### Example 4.6

### Immunogenicity—Induction of CD19⁺CD27⁺IL10⁺ B-cells

PBMCs isolated from allergic patients (AP, n=16) and non-atopic (NAC, n=6) individuals using flow cytometry. PBMCs were stimulated with different batches of peptide or native proteins at 0, 0.1, 0.3, 1, 3 & 10 μg/mL concentrations for 72 hrs at 37° C. Cells were stimulated with PMA, Ionomycin and BFA (Brefeldin A) and incubated for a total of 5 hrs at 37° C. Following incubation, cells were immunostained with CD19, CD27 for 30 min at room temperature. Cells were fixed and permeabilized using Cytofix/cytoperm reagent for 20 min at 4° C. and immunostained with IL10 for 30 min. Cells were washed and resuspended in cell staining buffer before acquisition on the BD FACS Canto II instrument.

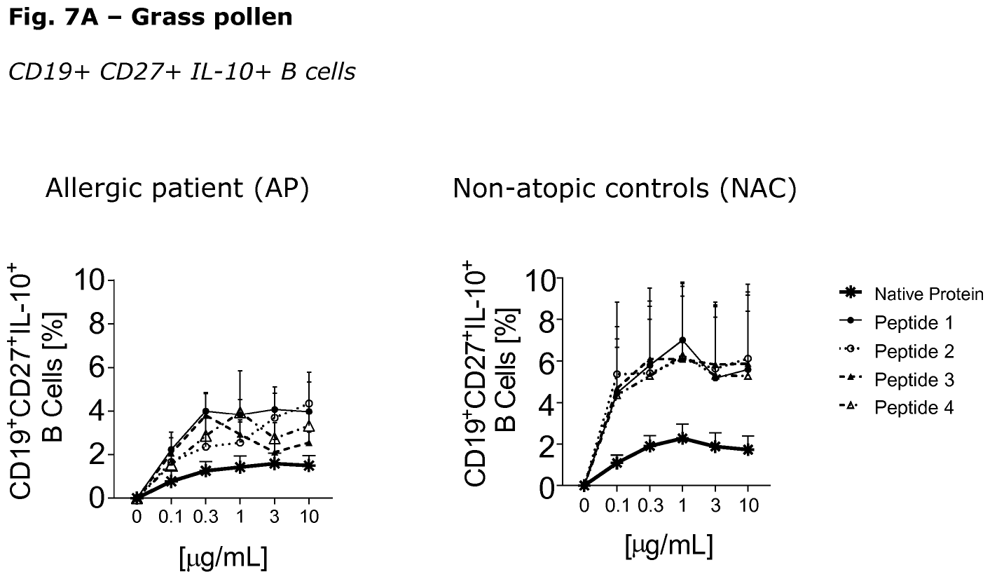

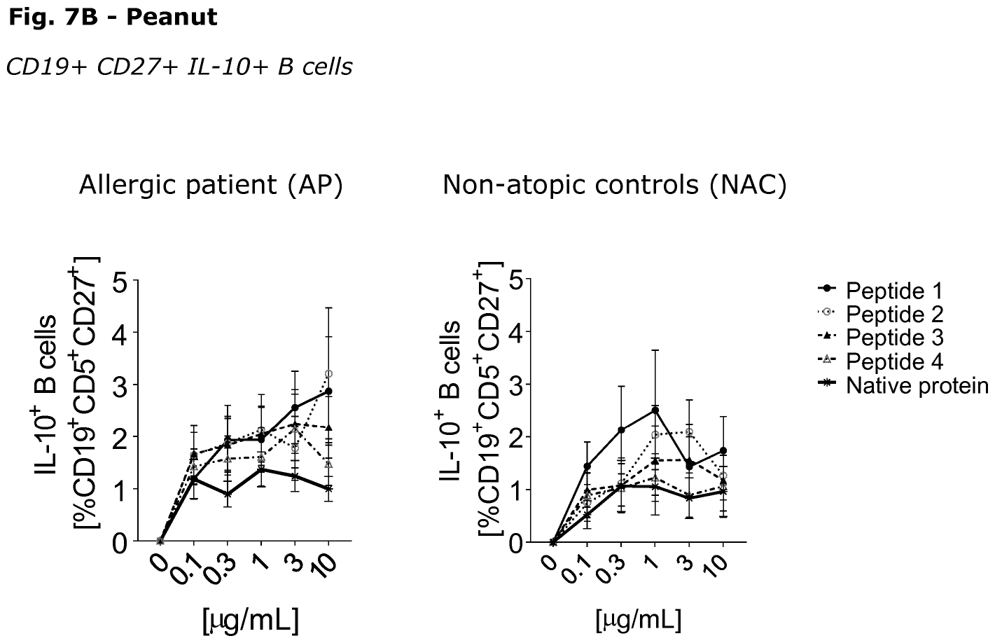

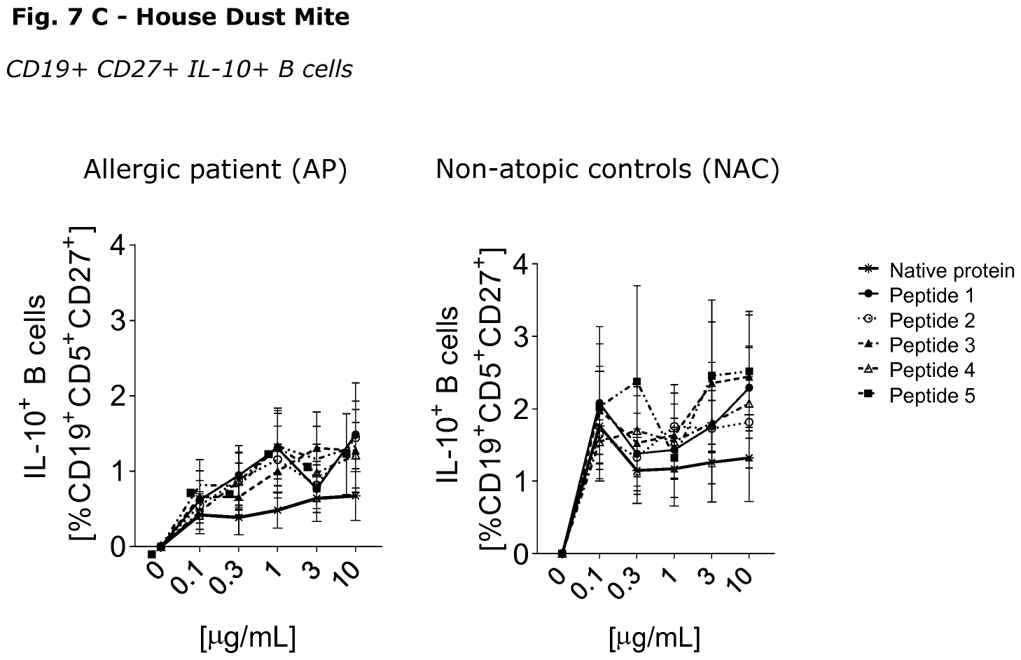

FIG. 7 shows results for grass pollen (Lolium perenne) (A), peanut (Arachis hypogaea) (B), and house dust mite (Dermatophagoides pteronyssinus) (C) allergen fragments. The proportion of CD19⁺CD27⁺IL10⁺ B-cells were significantly increased after stimulation with peptide batches compared to native proteins in non-atopics and allergics. This increase was observed in a dose-dependent manner. The magnitude of increase was most profound in non-atopics.

### Example 4.7

### Immunogenicity—Induction of CD19⁺CD5⁺CD38^{hi}CD24^{hi}IL10⁺ B-cells

PBMCs isolated from allergic patients (AP, n=16) and non-atopic (NAC, n=6) individuals using flow cytometry. PBMCs were stimulated with different batches of peptides or native proteins at 0, 0.1, 0.3, 1, 3 & 10 μg/mL concentrations for 72 hrs at 37° C. Cells were stimulated with PMA, Ionomycin and BFA (Brefeldin A) and incubated for a total of 5 hrs at 37° C. Following incubation, cells were immunostained with CD19, CD5, CD38, CD24 for 30 min at room temperature. Cells were fixed and permeabilized using Cytofix/cytoperm reagent for 20 min at 4° C. and immunostained with IL10 for 30 min. Cells were washed and resuspended in cell staining buffer before acquisition on the BD FACS Canto II instrument.

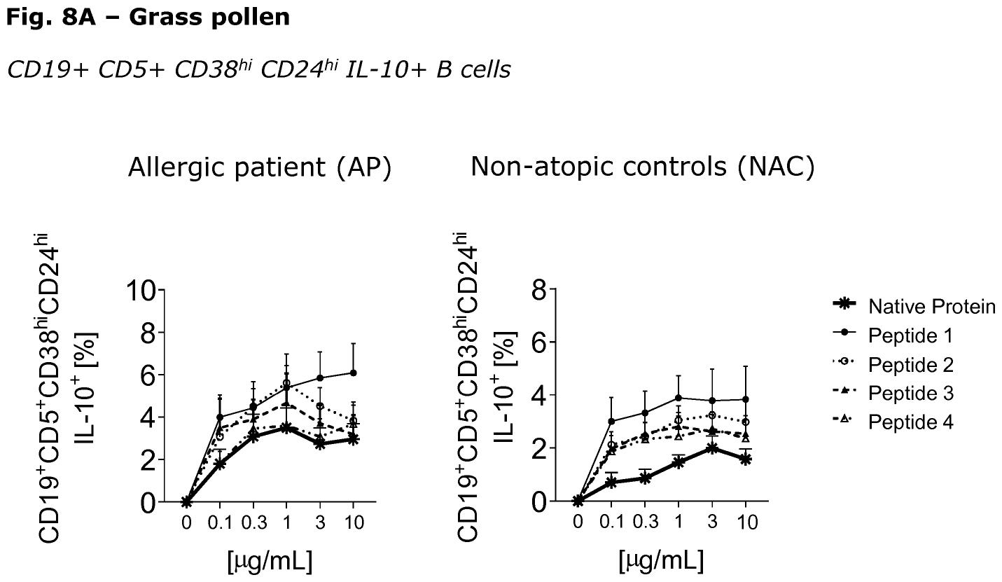

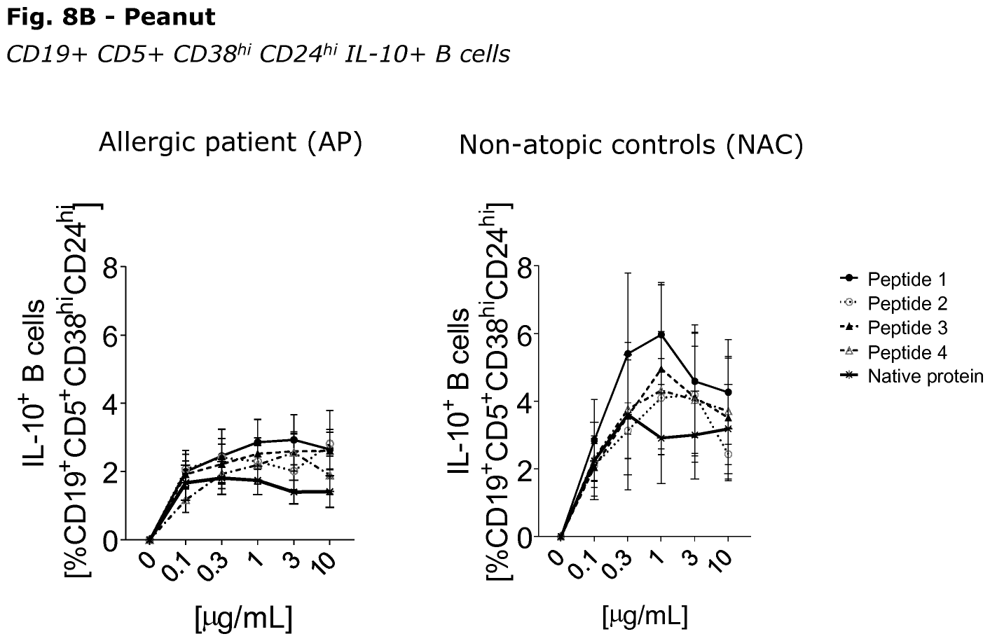

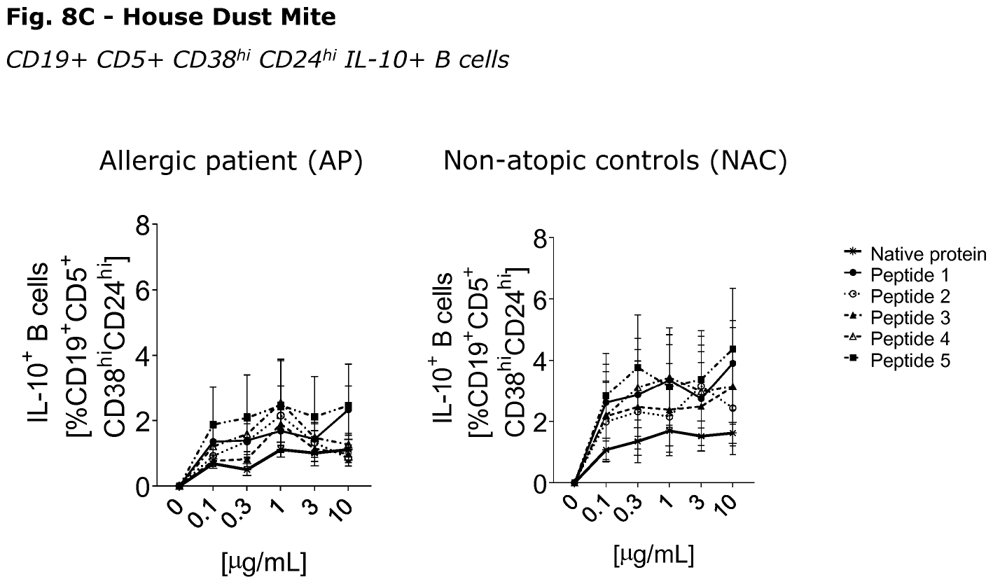

FIG. 8 shows results for grass pollen (Lolium perenne) (A), peanut (Arachis hypogaea) (B), and house dust mite (Dermatophagoides pteronyssinus) (C) allergen fragments. The proportion of CD19⁺CD5⁺CD38^{hi}CD24^{hi}B-cells in PBMCS were significantly increased following stimulation with peptide batches compared to native proteins in non-atopics. This increase was observed in a dose-dependent manner. Moreover, the proportion of CD19⁺CD5⁺CD38hiCD24hi B-cells in PBMCS were significantly increased after stimulation with peptide batches compared to native proteins in allergic subjects.

### Example 4.8

### Immunogenicity—Induction of CD19⁺CD5⁺CD38^{int}CD24^{int}IL10⁺ B-cells

PBMCs isolated from allergic patients (GPA, n=16) and non-atopic (NAC, n=6) individuals using flow cytometry. PBMCs were stimulated with different peptide batches or native proteins at 0, 0.1, 0.3, 1, 3 & 10 μg/mL concentrations for 72 hrs at 37° C. Cells were stimulated with PMA, Ionomycin and BFA (Brefeldin A) and incubated for a total of 5 hrs at 37° C. Following incubation, cells were immunostained with CD19, CD5, CD38, CD24 for 30 min at room temperature. Cells were fixed and permeabilized using Cytofix/cytoperm reagent for 20 min at 4° C. and immunostained with IL10 for 30 min. Cells were washed and resuspended in cell staining buffer before acquisition on the BD FACS Canto II instrument.

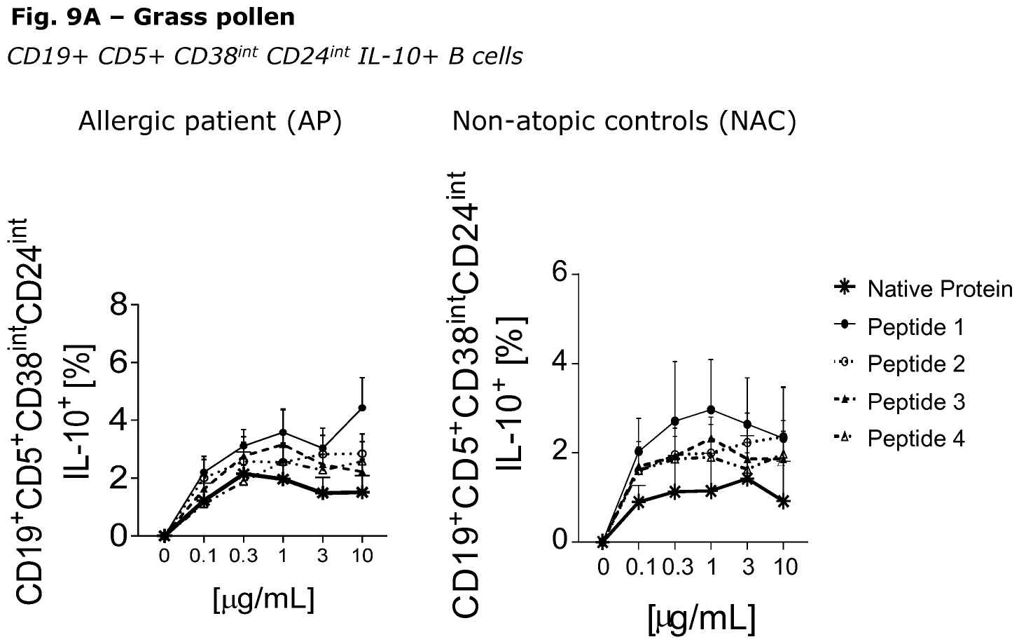

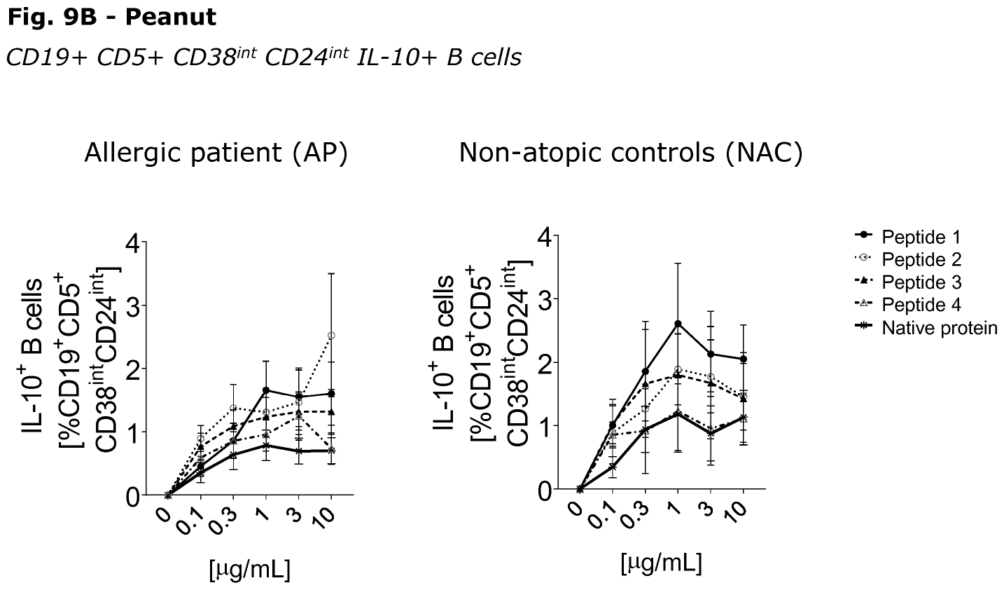

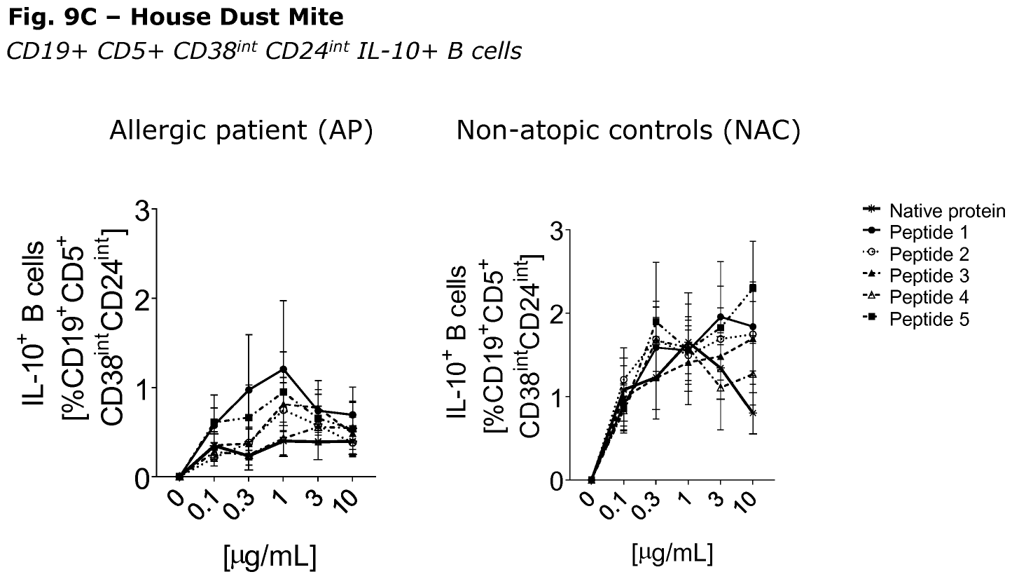

FIG. 9 shows results for grass pollen (Lolium perenne) (A), peanut (Arachis hypogaea) (B), and house dust mite (Dermatophagoides pteronyssinus) (C) allergen fragments. The proportion of CD19⁺CD5⁺CD38^{int}CD24^{int} B-cells in PBMCS were significantly increased following stimulation with peptide batches compared to native proteins in non-atopics. This increase was observed in a dose-dependent manner. Moreover, the proportion of CD19⁺CD5⁺CD38^{hi}CD24^{hi} B-cells in PBMCS were significantly increased after stimulation with peptide batches compared to native proteins in allergic subjects.

1. A method for the evaluation of a hydrolyzed allergen preparation comprising the steps of:
bringing the preparation into contact with a human blood sample measuring proliferation of IL10 producing regulatory B-cells, wherein proliferation indicates suitability of the preparation.

2. The method of claim 1, wherein the method is for the evaluation of the suitability of the hydrolyzed allergen preparation for the treatment or prevention of IgE mediated allergy.

3. The method of claim 1, wherein tec cation is a quality control within a production process.

4. The method of claim 1, wherein the evaluation is a screening in drug development.

5. The method of claim 1, wherein the method further comprises:
bringing an unhydrolyzed allergen preparation into contact with a second identical blood sample
measuring proliferation of IL10 producing regulatory B-cells comparing proliferation in the two samples,
wherein a higher proliferation in the sample being in contact with the hydrolyzed allergen preparation than in the sample in contact with the unhydrolyzed allergen indicates suitability.

6. The method of claim 1, wherein the blood sample is from a subject being allergic to the allergen.

7. The method of claim 1, wherein the blood sample is from a subject being non-allergic to the allergen.

8. The method of claim 1, wherein the allergens is selected from allergens are selected among pollen allergens, milk allergens, venom allergens, egg allergens, weed allergens, grass allergens, tree allergens, shrub allergens, flower allergens, vegetable allergens, grain allergens, fungi allergens, fruit allergens, berry allergens, nut allergens, seed allergens, bean allergens, fish allergens, shellfish allergens, seafood allergens, meat allergens, spices allergens, insect allergens, mite allergens, mould allergens, animal allergens, pigeon tick allergens, worm allergens, soft coral allergens, animal dander allergens, nematode allergens, allergens of Hevea brasiliensis.

9. The method of claim 1, wherein the method comprises steps for preparing the hydrolyzed allergens:
a) extracting a natural source of allergens comprising allergenic proteins to form an extract,
b) purifying of said extract to remove non-protein components to form a purified extract,
c) denaturing said purified extract to form a purified denatured extract,
d) hydrolyzing the purified denatured extract to form hydrolyzed allergen peptides.

10. The method of claim 1, wherein the method comprises steps for preparing the hydrolyzed allergens
a) extracting a source of allergens comprising allergenic proteins to fort an extract,
b) purifying the extract to remove non-protein components to form a purified extract,
c) denaturing the purified extract with a first denaturing agent to form a purified denatured extract,
d) refining the purified denatured e trac to remove impurities to form a refined denatured extract,
e) denaturing the refined denatured extract with a second denaturing agent to form denatured allergen mixture, and
f) hydrolyzing the denatured allergen mixture to form the hydrolyzed allergen peptides.

11. The method of claim 1, wherein the IL10 producing regulatory B-cells are CD19³⁰ IL10⁺ B-cells.

12. The method of claim 1, wherein the IL10 producing regulatory B-cells are CD19⁺CD27⁺IL10⁺ B-cells.

13. The method of claim 1, wherein the IL10 producing regulatory B-cells are CD19⁺CD5⁺CD38^{hi} CD24^{hi}IL10⁺ B-cells.

14. The method of claim 1, wherein the IL10 producing regulatory B-cells are CD19⁺CD5⁺CD38^{int}CD24^{int}IL10⁺ B-cells.
